SEQUENCE LISTING (12 sequences)

SEQ ID NO: 1 - DNA, 5518 bp, Artificial
  recombinant plasmid p-LC
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagagtcgacaaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgcctatgagtgtgctcactcaggtcctggcgttgctgctgctgtggcttacaggtacgcgttgtgacatcgtgatgacccagtctccatcctccctgtctgcatctgtaggagacagagtcaccatcacttgccgggcaagtcagaacattatccgctatttaaattggtatcagcagaagccagggaaagcccctaggctcctgatctatggtgcgtccactttgcaaagtggggtcccatcaaggttcagtggcagtggatctgggacagatttcactctcaccatcagtagtctgcaacctgaagattttgcaacttactactgtcaacagagttaccgtacccctccattcactttcggccctgggaccaagcttgaaatcaaacctgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagaagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagattctagagtcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccccgatagacggtttttcgccctttgacgctggagttcacgttcctcaatagtggactcttgttccaaactggaacaacactcaaccctatctcggtctattcttttgatttataagggatttttccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 2 - protein, 234 aa, Artificial
  Synthetic Construct
MSVLTQVLALLLLWLTGTRCDIVMTQSPSSLSASVGDRVTITCRASQNIIRYLNWYQQKPGKAPRLLIYGASTLQSGVPSRFSGSGSGTDFTLTISSLQPEDFATYYCQQSYRTPPFTFGPGTKLEIKPVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - DNA, 6238 bp, Artificial
  recombinant plasmid p-HC
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagagtcgacaaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccatggcttgggtgtggaccttgccattcctgatggcagctgcccaaggtgtcgacgcacaggtgaaactgctcgagtctgggggaggcgtggtccagccgggggggtccctgagactctcctgtgaagcgtctggattcgccctcagaagttctggcatgcactgggtccgccaggctcctggcaaggggctggagtgggtggcacttatatggtttgatggaagtatcagatcgtatgcagaatccgtgaagggccgattcaccatctccagagacacttccaagaacaccctatatctccaaatgcgcagtctgagtgccgacgacacggctgtgtattactgtgcgagagacaaggcggttcggggaattagcaggtacaactattacatggacgtctggggcaaagggaccacggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatgataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagattctagagtcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccccgatagacggtttttcgccctttgacgctggagttcacgttcctcaatagtggactcttgttccaaactggaacaacactcaaccctatctcggtctattcttttgatttataagggatttttccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 4 - protein, 474 aa, Artificial
  Synthetic Construct
MAWVWTLPFLMAAAQGVDAQVKLLESGGGVVQPGGSLRLSCEASGFALRSSGMHWVRQAPGKGLEWVALIWFDGSIRSYAESVKGRFTISRDTSKNTLYLQMRSLSADDTAVYYCARDKAVRGISRYNYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - DNA, 5291 bp, Artificial
  recombinant plasmid p-p18
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccatggccgagccttgggggaacgagttggcgtccgcagctgccaggggggacctagagcaacttactagtttgttgcaaaataatgtaaacgtcaatgcacaaaatggatttggaaggactgcgctgcaggttatgaaacttggaaatcccgagattgccaggagactgctacttagaggtgctaatcccgatttgaaagaccgaactggtttcgctgtcattcatgatgcggccagagcaggtttcctggacactttacagactttgctggagtttcaagctgatgttaacatcgaggataatgaagggaacctgcccttgcacttggctgccaaagaaggccacctccgggtggtggagttcctggtgaagcacatggccagcaatgtggggcatcggaaccataagggggacaccgcctgtgatttggccaggctctatgggaggaatgaggttgttagcctgatgcaggcaaacggggctgggggagccacaaatcttcaataataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccccgatagacggtttttcgccctttgacgctggagttcacgttcctcaatagtggactcttgttccaaactggaacaacactcaaccctatctcggtctattcttttgatttataagggatttttccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 6 - protein, 168 aa, Artificial
  Synthetic Construct
MAEPWGNELASAAARGDLEQLTSLLQNNVNVNAQNGFGRTALQVMKLGNPEIARRLLLRGANPDLKDRTGFAVIHDAARAGFLDTLQTLLEFQADVNIEDNEGNLPLHLAAKEGHLRVVEFLVKHMASNVGHRNHKGDTACDLARLYGRNEVVSLMQANGAGGATNLQ

SEQ ID NO: 7 - DNA, 5279 bp, Artificial
  recombinant plasmid p-p21
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccatgtcagaaccggctggggatgtccgtcagaacccatgcggcagcaaggcctgccgccgcctcttcggcccagtggacagcgagcagctgagacgcgactgtgatgcgctaatggcgggctgcatccaggaggcccgtgagcgatggaacttcgactttgtcaccgagacaccactggagggtgacttcgcctgggagcgtgtgcggggccttggcctgcccaagctctaccttcccacggggccccggcgaggccgggatgagttgggaggaggcaggcggcctggcacctcacctgctctgctgcaggggacagcagaggaagaccatgtggacctgtcactgtcttgtacccttgtgcctcgctcaggggagcaggctgaagggtccccaggtggacctggagactctcagggtcgaaaacggcggcagaccagcatgacagatttctaccactccaaacgccggctgatcttctccaagaggaagccctaataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccccgatagacggtttttcgccctttgacgctggagttcacgttcctcaatagtggactcttgttccaaactggaacaacactcaaccctatctcggtctattcttttgatttataagggatttttccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 8 - protein, 164 aa, Artificial [sequence omitted]

SEQ ID NO: 9 - DNA, 5252 bp, Artificial
  recombinant plasmid p-aFGF
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccatggctgaaggggaaatcaccaccttcacagccctgaccgagaagtttaatctgcctccagggaattacaagaagcccaaactcctctactgtagcaacgggggccacttcctgaggatccttccggatggcacagtggatgggacaagggacaggagcgaccagcacattcagctgcagctcagtgcggaaagcgtgggggaggtgtatataaagagtaccgagactggccagtacttggccatggacaccgacgggcttttatacggctcacagacaccaaatgaggaatgtttgttcctggaaaggctggaggagaaccattacaacacctatatatccaagaagcatgcagagaagaattggtttgttggcctcaagaagaatgggagctgcaaacgcggtcctcggactcactatggccagaaagcaatcttgtttctccccctgccagtctcttctgattaataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccccgatagacggtttttcgccctttgacgctggagttcacgttcctcaatagtggactcttgttccaaactggaacaacactcaaccctatctcggtctattcttttgatttataagggatttttccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 10 - protein, 155 aa, Artificial
  Synthetic Construct
MAEGEITTFTALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 11 - DNA, 6196 bp, Artificial
  recombinant plasmid p-Enbrel
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagagtcgacaaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccatggcgcccgtcgccgtctgggccgcgctggccgtcggactggagctctgggctgcggcgcacgccttgcccgcccaggtggcatttacaccctacgccccggagcccgggagcacatgccggctcagagaatactatgaccagacagctcagatgtgctgcagcaaatgctcgccgggccaacatgcaaaagtcttctgtaccaagacctcggacaccgtgtgtgactcctgtgaggacagcacatacacccagctctggaactgggttcccgagtgcttgagctgtggctcccgctgtagctctgaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggaactgaaacatcagacgtggtgtgcaagccctgtgccccggggacgttctccaacacgacttcatccacggatatttgcaggccccaccagatctgtaacgtggtggccatccctgggaatgcaagcatggatgcagtctgcacgtccacgtcccccacccggagtatggccccaggggcagtacacttaccccagccagtgtccacacgatcccaactcgacgttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatgataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagattctagagtcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccccgatagacggtttttcgccctttgacgctggagttcacgttcctcaatagtggactcttgttccaaactggaacaacactcaaccctatctcggtctattcttttgatttataagggatttttccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 12 - protein, 460 aa, Artificial
  Synthetic Construct
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQLDVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2008/0145893 A1
(43) Pub. Date: Jun. 19, 2008

(54) Method for producing a recombinant protein at high specific productivity, high batch yield and high volumetric yield by means of transient transfection

(75) Inventors: Markus Hildinger, Zurich (CH); Sarah Wulhfard, Fernev Voltaire (IT); Gaurav Backliwal, Lausanne (CH); David Hacker, Nyon (CH); Maria De Jesus, Chavannes (CH); Florian Maria Wurm, Le Mont-sur-Lausanne (CH)

Correspondence Address: MARKUS HILDINGER, CRANACHWEG 8, PFORZHEIM, 75173, omitted

(73) Assignee: EXCELLEGENE SA, Monthey (CH)

(21) Appl. No.: 11/309,732

(22) Filed: Sep. 17, 2006

(51) Int. Cl. C12P 21/06; C12N 15/09; C12N 5/06; C07K 14/50

(52) U.S. Cl. 435/69.1; 435/455; 435/325; 530/399

(57) ABSTRACT

Recombinant proteins are of great commercial interest. Yet, most current production methods in mammalian cells involve the time- and labor-consuming step of creating stable cell lines. Production methods based on transient gene expression are advantageous in terms of speed and versatility, yet, thus far, those methods have not shown the specific productivity, batch yield and volumetric yield to be an economic alternative to stable cell lines. The inventors improved on the methodology of transient transfection and achieved commercially relevant yields in terms of specific productivity (exceeding 35 pg per cell per day), batch yield (exceeding 700 mg/l) and volumetric yield.

### INCORPORATION-BY-REFERENCE OF MATERIAL SUBMITTED ON A COMPACT DISC

### Reference to Sequence Listing

A paper copy of the Sequence Listing and a computer readable form (CRF) of the sequence listing, containing the file named “transient tx.ST25.txt” which is 75.4 kilobytes in size, and which was created on Sep. 17, 2006 and last modified on Sep. 17, 2006, are herein incorporated by reference. The symbols and format used for nucleotide and amino acid sequence data comply with the rules set forth in 37 C.F.R. section 1.822.

### BACKGROUND OF THE INVENTION

It must be noted that as used herein and in the appended claims, the singular forms “a” and “the” include plural referents unless the context clearly dictates otherwise. Thus, for example, reference to “a cell” or “the cell” includes a plurality (“cells” or “the cells”), and so forth. Moreover, the word “or” can either be exclusive in nature (i.e., either A or B, but not A and B together), or inclusive in nature (A or B, including A alone, B alone, but also A and B together). One of skill in the art will realize which interpretation is the most appropriate unless it is detailed by reference in the text as “either A or B” (exclusive “or”) or “and/or” (inclusive “or”).

A portion of the disclosure of this patent document contains material which is subject to copyright protection. The copyright owner has no objection to the facsimile reproduction by anyone of the patent document or the patent disclosure as it appears in the Patent and Trademark Office file or records, but otherwise reserves all copyright rights whatsoever.

(1) Field of the Invention

The present invention relates to the field of biotechnology in general, and to methods for producing recombinant proteins in mammalian cells in particular.

In another aspect, the present invention relates to methods for producing recombinant proteins in mammalian cells by means of transient gene expression.

In another aspect, the present invention relates to methods for producing recombinant proteins in mammalian cells by means of transfection of nucleic acids into mammalian cells. In one embodiment, transfection of nucleic acids into mammalian cells is achieved by using polyethyleneimine as transfection reagent.

In yet another aspect, the present invention relates to the production of recombinant proteins in mammalian cells, where said recombinant proteins are antibodies, Fc-tagged fusion proteins, growth factors or any other protein of commercial interest.

In yet another aspect, the present invention relates to an alternative to currently existing production methods for recombinant protein production with batch yields exceeding 200 mg/l. Current, existing methods for production of recombinant proteins with batch yields exceeding 200 mg/l are primarily based on the generation and selection of stable cell lines. The present invention does not require the generation and selection of stable cell lines, but allows for high titer (>200 mg/l) production of recombinant proteins by means of transient gene expression by means of nucleic acid transfection.

In yet another aspect, the present invention relates to the field of transfection of nucleic acids into mammalian cells. In one aspect, the present invention teaches how to achieve high transfection efficiency in terms of the number of nucleic acid molecules (e.g., plasmids) transferred into mammalian cells. Some of those teachings include transfection of mammalian cells at high cell densities, transfection of mammalian cells by directly adding the DNA and the transfection reagent to the cells, and by teaching the optimal time until diluting the transfection mixture.

In yet another aspect, the present invention relates to the field of extending viability of mammalian cell cultures. In one aspect, this relates to the field of recombinant protein production. The present invention teaches methods of how to extend the viability of mammalian cell cultures, e.g., by adding sodium butyrate, by shifting the temperature into a range of 28° C. to 32° C., by co-expressing cell-cycle regulatory proteins such as p18 or p21.

(2) Description of Related Art

Many single aspects of the present invention have been described in prior art, including transfection of mammalian cells such as HEK293 or CHO cells, expression of p18 and p21 to improve batch yields, the use of sodium butyrate to improve batch yields, the use of temperature shift to improve batch yields, the use of polyethyleneimine to transfect mammalian cells, the use of 2-aminopurine to improve batch yields.

Yet, yields of prior art methods of transient transfection are rather disappointing with no description of yields exceeding 100 mg/l or even 200 mg/l of recombinant protein in general and antibodies in particular. The inventors are the first to demonstrate specific productivity exceeding 20 pg per cell per day and even 35 pg per cell per day as well as batch yields exceeding 200 mg/l or even 700 mg/l.

Thus, the merits of the present invention are based on the improvement and combination of individual methods described in prior art. Only through the novel and non-obvious combination and improvement of prior art methods the batch yields and specific productivity as disclosed in the present invention can be achieved. These aspects are discussed in more detail in the paragraphs summarizing the novelty of the present invention.

### BRIEF SUMMARY OF THE INVENTION

### (1) Substance or General Idea of the Claimed Invention

Recombinant proteins are of great commercial interest. Yet, most current production methods in mammalian cells involve the time- and labor-consuming step of creating stable cell lines. Production methods based on transient gene expression are advantageous in terms of speed and versatility; yet, thus far, those methods have not shown the specific productivity, batch yield and volumetric yield to become an economic alternative to stable cell lines. The inventors have improved on the methodology of transient transfection and achieved commercially relevant yields in terms of specific productivity (exceeding 35 pg per cell per day), batch yield (exceeding 700 mg/l) and volumetric yield.

In particular, the inventors have improved the method of recombinant protein production by transient gene expression through the combination and modification of individual methods described in prior art. Only through the novel and non-obvious combination and improvement of prior art methods the batch yields, volumetric yields and specific productivity as disclosed in the present invention can be achieved.

In one aspect, the inventors teach how to obtain high transfection efficiency in terms of numbers of plasmids transferred into mammalian cells. High transfection efficiency is a prerequisite to obtain high specific productivity, high batch yields and high volumetric yields. For example, the inventors teach optimal transfection efficacy requires transfection at high cell densities (e.g., of approximately 20 million cells per ml). Furthermore, transfection efficacy is increased by directly adding the nucleic acids and the transfection reagent to the cells—without prior, separate incubation of the nucleic acids and the transfection reagent. In addition, optimal transfection at high cell densities requires dilution of the transfection mixture (comprising the cells, the DNA and the transfection reagent) at a certain time point to a certain cell density. The present invention also teaches how to achieve this aspect. Last, but not least, optimal transfection efficacy also requires the optimal amount and ratio of nucleic acids to transfection reagent, which is taught as well by the present invention.

In another aspect, the inventors teach how to obtain high specific productivity, batch yields and high volumetric yields by co-transfecting and co-expressing auxiliary proteins. These auxiliary proteins comprise p18, p21 and aFGF. Whereas prior art describes inducible systems for p21 expression, there are no prior art descriptions detailing p18 and p21 coexpression to increase specific productivity, batch yields and volumetric yields in the context of transient gene expression. Furthermore, there are no prior art references on the usefulness of combining p18 and p21 coexpression with aFGF coexpression.

In yet another aspect, the inventors teach how to obtain high batch yields and high volumetric yields by extending the culture viability. All else equal, a culture that is viable for 14 days should yield higher batch yields and higher volumetric yields than a culture that is viable for only 5 days (assuming similar specific productivity). The inventors teach several different methods on how to achieve elongated culture viability in the context of transient gene expression, e.g., by adding sodium butyrate or by shifting the temperature to around 31° C. or by co-expressing cell-cycle regulatory proteins (e.g., p18, p21). These methods are only examples, and any method capable of extending the culture viability should fall within the scope of the present invention.

In another aspect, the present invention relates to the production of recombinant proteins at the 10 liter scale by using non-instrumented “bioreactors”, e.g., by using non-instrumented plastic vessels that are shaken in an orbital shaker.

To summarize the most important teachings of the present invention: Whereas many single aspects are crucial to obtain high batch yields, high specific productivity and high volumetric yields based on transient gene expression, the three most crucial ones are the combination of

(1) high transfection efficacy (achieved by transfecting at high cell densities of optimally around 20 million cells per ml and by directly adding the nucleic acids and the transfection reagent to the mammalian cells and by using the optimal amount and ratio of nucleic acids to transfection reagent and by diluting the transfection mix at the optimal time point);
(2) extended cell culture viability (up to approximately 20 days; achieved by adding sodium butyrate, temperature shift, and/or coexpression of p18 or p21);
(3) optimized cell density after transfection (achieved by diluting the cells to a density of 2 million cells per ml up to 5 million cells per ml).
(4) It should also be noted that optimal transfection efficacy without extending cell culture viability does not result in high batch yields. Particularly, when using HEK293 cells, it is crucial to extend cell culture viability and to prevent the cells from continuing to divide in order to preserve cell viability and to achieve high batch yields. This is contrary to current wisdom, where people tend to expand the cell culture during the production phase. According to the teachings of the current invention as it relates to HEK293 cells, cells should be expanded prior to transfection, but optimally should not divide any more after transfection.

### (2) Advantages of the Invention Over Prior Approaches

Usefulness of the Present Invention

Recombinant proteins are of great commercial and scientific interest. Yet, most current production methods in mammalian cells involve the time- and labor-consuming step of creating stable cell lines. Production methods based on transient gene expression are advantageous in terms of speed and versatility; yet, thus far, those methods have not shown the specific productivity, batch yield and volumetric yield to be an economic alternative to stable cell lines. The inventors improved on the methodology of transient transfection and achieved commercially relevant yields in terms of specific productivity (exceeding 35 pg per cell per day), batch yield (exceeding 700 mg/l) and volumetric yield.

The method of the present invention—based on production of recombinant proteins by means of transient transfection—will prove useful in many aspects where traditional technology based on the establishment of stable recombinant cell lines faces difficulties. Some examples include:
(1) Production of cell-toxic proteins. Toxic proteins prevent the establishment of a cell clone as well as expansion of cell clones. Thus, toxic proteins cannot be produced by traditional methods based on the establishment of a recombinant cell line. Here, transient transfection offers a solution as cells can be expanded prior to transfection (in the absence of the toxic protein), and the toxic protein can be produced upon transfection (for as long as the cells survive in the presence of the toxic protein).
(2) Production of proteins that interfere with cell division. Similar to cell-toxic proteins, expression of proteins that inhibit cell division do not allow for the establishment of stable recombinant cell clones. Here, transient transfection offers a solution. And as cell division after transfection is not required for protein production (as the cells are expanded prior to transfection), proteins that inhibit cell division can be produced in large quantities by means of transient transfection.
(3) Fast production of recombinant proteins. The current method based on the establishment of recombinant cell lines is rather time consuming: The time from transfection via clone selection to production can take from 3 to 6 months or even longer. With transient transfection, the time from transfection to the final product does not take more then two to three weeks. This could be of relevance in situations where large quantities of proteins are required in a short amount of time, e.g., to produce a vaccine, to speed up clinical development, etc.
(4) Production of a broad set of recombinant proteins. The current method based on the establishment of recombinant cell lines is rather labor intensive (clone picking, clone screening, clone expansion) and thus, not easy to scale up (apart from requiring a significant amount of time from transfection to the final product). Furthermore, one would need to preserve each clone in order not to lose the cell line. For example, if one wanted to express 1,000 different proteins, one would have to establish and keep in culture (or store) 1,000 different cell lines. Assuming a screening of 100 clones per cell line, one would have to screen a total of 100,000 clones. With transient transfection, one would need to have only one cell line in culture (the one to be transfected) and does not have to store individual clones, but can just retransfect the cells in order to produce a certain recombinant protein a second time. This advantage is useful in the context of creating large libraries of diverse proteins, for example in the context of screening large protein libraries for their pharmaceutical usefulness.

Whereas prior art methods of transient transfection can be used to address the aforementioned issues, yields of prior art methods of transient transfection for the production of recombinant proteins are rather disappointing with no description of yields exceeding 100 mg/l or even 200 mg/l. The inventors are the first to demonstrate specific productivity exceeding 20 pg per cell per day and even 35 pg per cell per day as well as batch yields exceeding 200 mg/l or even 700 mg/l. Only at the yields obtained by the inventors for the first time, the applications mentioned above become economically viable and scientifically interesting. Thus, the usefulness of the present invention comprises both

(1) the flexibility and speed advantages offered by transient transfection, and

(2) yields close to those obtained with average stable cell lines.

Novelty of the Present Invention

Methods for transient transfection have been described in prior art—including transient transfection using 25-kd linear polyethyleneimine as transfection reagent. Yet, yields obtained with prior art methods for transient transfection have not exceeded 100 mg/l or 200 mg/l or 700 mg/l, and specific productivity has not reached 20 pg/cell/day or 35 pg/cell/day.

The inventors are the first to describe a method of transient transfection yielding a specific productivity exceeding 20 pg/cell/day and even 35 pg/cell/day and batch yields exceeding 200 mg/l and even 700 mg/l. Furthermore, the inventors achieved those yields for the recombinant production of therapeutic antibodies, which belong the group of proteins most difficult to produce by means of recombinant technology.

Thus, the novelty of the present invention is based on the high specific productivity and batch yields obtained by the method developed by the inventors, which has not yet been demonstrated in prior art.

Non-Obviousness of the Present Invention

Whereas methods of transient transfections have been described, no transient transfection described in prior art has achieved the specific productivity and the batch yields obtained with the method described in the present invention. Given the importance of high yields and specific productivity for commercial applications as well as scientific research, the artisan would have preferred a method with higher yields compared to a method with lower yields (all else equal) if it were obvious how to use such a method. For example, why to produce a specific protein at a 200 liter scale if one could produce the same amount at a 10 liter scale by optimizing the transient transfection protocol—especially as for many laboratories, the 200 liter scale is beyond their technological equipment?

Furthermore, whereas many single aspects of the present invention have been described in diverse contexts, it is the combination of those different aspects and improvement and modification of those different aspects that are required to obtain the batch yields and specific productivity as described in the present invention, and the combination of those diverse aspects is not obvious; equally, the optimized combination as described in the present invention cannot be obtained without undue effort. For example, the use of sodium butyrate to increase titers has been described in prior art as well as transient transfection using 25-kd PEI as transfection reagent. Yet, the optimized cell density at the time of transfection as well as the optimized PEI-to-DNA ratio as well as the timing and quantity of sodium butyrate addition as well as the co-transfection of a plasmid encoding aFGF is neither obvious nor has the combination been described in prior art.

Besides, some of the findings of the present invention, which are crucial to obtaining high batch yields and high specific productivity are contradictory to current wisdom and thus not obvious to one of ordinary skill in the art. For example, to obtain maximum yields, it is important to transfect the cells at extremely high cell densities (up to 20 million cells per ml). No transfection protocol has been described that demands such high cell densities. Moreover, in the preferred embodiment, the DNA is directly added to the cells being transfected, i.e., there is no pre-DNA-transfection reagent complex formation. Most protocols—particularly those based on PEI transfection—demand a mixture of DNA with the transfection reagent prior to adding it to the cells. And current wisdom also suggests expanding cells after transfection for optimal yields (to increase the cell mass), whereas the teachings of the present invention suggest cell division/expansion after transfection to be detrimental to overall yields.

### DETAILED DESCRIPTION OF THE INVENTION

The practice of the present invention will employ, unless otherwise indicated, conventional methods of virology, microbiology, molecular biology and recombinant DNA techniques within the skill of the art. Such techniques are explained fully in the literature; see, e.g., Sambrook, et al. Molecular Cloning: A Laboratory Manual (Current Edition); DNA Cloning: A Practical Approach, vol. I & II (D. Glover, ed.); Oligonucleotide Synthesis (N. Gait, ed., Current Edition); Nucleic Acid Hybridization (B. Hames & S. Higgins, eds., Current Edition); Transcription and Translation (B. Hames & S. Higgins, eds., Current Edition); CRC Handbook of Parvoviruses, vol. I & II (P. Tijessen, ed.); Fundamental Virology, 2nd Edition, vol. I & II (B. N. Fields and D. M. Knipe, eds.)

### (1) DEFINITIONS

In describing the present invention, the following terms will be employed, and are intended to be defined as indicated below.

For purposes of this invention, the term “protein” means a polypeptide (native [i.e., naturally-occurring] or mutant), oligopeptide, peptide, or other amino acid sequence. As used herein, “protein” is not limited to native or full-length proteins, but is meant to encompass protein fragments having a desired activity or other desirable biological characteristics, as well as mutants or derivatives of such proteins or protein fragments that retain a desired activity or other biological characteristic including peptoids with nitrogen based backbone. Mutant proteins encompass proteins having an amino acid sequence that is altered relative to the native protein from which it is derived, where the alterations can include amino acid substitutions (conservative or non-conservative), deletions, or additions (e.g., as in a fusion protein). “Protein” and “polypeptide” are used interchangeably herein without intending to limit the scope of either term.

For purposes of this invention, “amino acid” refers to a monomeric unit of a peptide, polypeptide, or protein. There are twenty amino acids found in naturally occurring peptides, polypeptides and proteins, all of which are L-isomers. The term also includes analogs of the amino acids and D-isomers of the protein amino acids and their analogs.

For purposes of this invention, by “DNA” is meant a polymeric form of desoxyribonucleotides (adenine, guanine, thymine, or cytosine) in double-stranded or single-stranded form, either relaxed or supercoiled, either linear or circular. This term refers only to the primary and secondary structure of the molecule, and does not limit it to any particular tertiary forms. Thus, this term includes single- and double-stranded DNA found, inter alia, in linear DNA molecules (e.g., restriction fragments), viruses, plasmids, and chromosomes. In discussing the structure of particular DNA molecules, sequences may be described herein according to the normal convention of giving only the sequence in the 5′ to 3′ direction along the non-transcribed strand of DNA (i.e., the strand having the sequence homologous to the mRNA). The term captures molecules that include the four bases adenine (A or a), guanine (G or g), thymine (T or t), or cytosine (C or c), as well as molecules that include base analogues which are known in the art.

For purposes of this invention, “polynucleotide” as used herein means a polymeric form of nucleotides of any length, either ribonucleotides or desoxyribonucleotides. This term refers only to the primary structure of the molecule. Thus, the term includes double- and single-stranded DNA, as well as, double- and single-stranded RNA. It also includes modifications, such as methylation or capping, and unmodified forms of the polynucleotide.

For the purpose of describing the relative position of nucleotide sequences in a particular nucleic acid molecule throughout the instant application, such as when a particular nucleotide sequence is described as being situated “upstream,” “downstream,” “5′,” or “3′” relative to another sequence, it is to be understood that it is the position of the sequences in the non-transcribed strand of a DNA molecule that is being referred to as is conventional in the art.

For purposes of this invention, a “gene sequence” or “coding sequence” or “protein coding sequence” or “open reading frame” or “cDNA” or a sequence which “encodes” a particular protein, is a nucleic acid composition which is transcribed into RNA (in the case of DNA) and potentially translated (in the case of mRNA) into a polypeptide in vitro or in vivo when placed under the control of appropriate regulatory control elements. The boundaries of the gene are determined by a start codon at the 5′ (amino) terminus and potentially a translation stop codon at the 3′ (carboxy) terminus. A gene sequence can include, but is not limited to, cDNA from prokaryotic or eukaryotic mRNA, genomic DNA sequences from prokaryotic or eukaryotic DNA, and even synthetic DNA sequences. A transcription termination sequence, which is a particular species of regulatory control element, will usually be located 3′ to the protein coding sequence.

For purposes of this invention, by the term “transgene” is meant a nucleic acid composition made out of DNA, which encodes a peptide, oligopeptide or protein. The transgene may be operatively linked to regulatory control elements in a manner which permits transgene transcription, translation and/or ultimately directs expression of a product encoded by the expression cassette in the producer cell, e.g., the transgene is placed into operative association with a promoter and enhancer elements, as well as other regulatory control elements, such as introns or polyA sequences, useful for its regulation. The composite association of the transgene with its regulatory sequences (regulatory control elements) is referred to herein as a “minicassette”, “expression cassette”, “transgene expression cassette”, or “minigene”. The exact composition of the expression cassette will depend upon the use to which the resulting (mini)gene transfer vector will be put and is known to the artisan (Sambrook 1989, Lodish et al. 2000). When taken up by a target cell, the expression cassette as part of the recombinant vector genome may remain present in the cell as a functioning extrachromosomal molecule, or it may integrate into the cell's chromosomal DNA, depending on the kind of transfer vector used. Generally, a minigene may have a size in the range of several hundred base pairs up to about 30 kb.

For purposes of this invention, “heterologous” as it relates to nucleic acid compositions denotes sequences that are not normally joined together. Thus, a “heterologous” region of a nucleic acid composition is a segment of nucleic acid within or attached to another nucleic acid composition that is not found in association with the other molecule in nature. For example, a heterologous region of a nucleic acid composition could include a coding sequence flanked by sequences not found in association with the coding sequence in nature. Another example of a heterologous coding sequence is a construct where the coding sequence itself is not found in nature (e.g., synthetic sequences having codons different from the native gene). Allelic variation or naturally occurring mutational events do not give rise to heterologous DNA, as used herein.

For purposes of this invention, “homology” or “homologous” refers to the percent homology between two polynucleotide moieties or two polypeptide moieties. The correspondence between the sequence from one moiety to another can be determined by techniques known in the art. Two DNA or two polypeptide sequences are “substantially homologous” to each other when at least about 80%, preferably at least about 90%, and most preferably at least about 95% of the nucleotides or amino acids match over a defined length of the molecules, as determined using methods in the art.

The techniques for determining amino acid sequence homology are well-known in the art. In general, “homology” (for amino acid sequences) means the exact amino acid to amino acid comparison of two or more polypeptides at the appropriate place, where amino acids are identical or possess similar chemical and/or physical properties such as charge or hydrophobicity. A so-termed “percent homology” then can be determined between the compared polypeptide sequences. The programs available in the Wisconsin Sequence Analysis Package (available from Genetics Computer Group, Madison, Wis.), for example, the GAP program, are capable of calculating homologies between two polypeptide sequences. In addition, the ClustalW algorithm is capable of performing a similar analysis. Other programs and algorithms for determining homology between polypeptide sequences are known in the art.

Homology for polynucleotides is determined essentially as follows: Two polynucleotides are considered to be “substantially homologous” to each other when at least about 80%, preferably at least about 90%, and most preferably at least about 95% of the nucleotides match over a defined length of the molecules, when aligned using the default parameters of the search algorithm BLAST 2.0. The BLAST 2.0 program is publicly available. The ClustalW algorithm can be utilized as well.

Alternatively, homology for polynucleotides can be determined by hybridization experiments. As used herein, a nucleic acid sequence or fragment (such as for example, primers or probes), is considered to selectively hybridize to a sequence 1, thus indicating “substantial homology”, if such a sequence is capable of specifically hybridizing to the sequence 1 or a variant thereof or specifically priming a polymerase chain reaction: (i) under typical hybridization and wash conditions, such as those described, for example, in Maniatis, (Molecular Cloning: A Laboratory Manual, 2nd Edition, 1989) where preferred hybridization conditions are those of lesser stringency and more preferred, higher stringency; or (ii) using reduced stringency wash conditions that allow at most about 25-30% base pair mismatches, for example, 2.times.SSC, 0.1% SDS, at room temperature twice, for 30 minutes each; then 2×SSC, 0.1% SDS, 37° C., once for 30 minutes; the 2×SSC at room temperature twice, 10 minutes each or (iii) under standard PCR conditions or under “touch-down” PCR conditions.

For purposes of this invention, the term “cell” means any prokaryotic or eukaryotic cell, either ex vivo, in vitro or in vivo, either separate (in suspension) or as part of a higher structure such as but not limited to organs or tissues.

For purposes of this invention, the term “host cell” means a cell that can be transduced and/or transfected by an appropriate gene transfer vector. The nature of the host cell may vary from gene transfer vector to gene transfer vector.

For purposes of this invention, the term “producer cell” means a cell that is capable of producing a recombinant protein. The producer cell itself may be selected from any mammalian cell. Particularly desirable producer cells are selected from among any mammalian species, including, without limitation, cells such as HEK 293, A549, WEHI, 3T3, 10T1/2, BHK, MDCK, COS 1, COS 7, BSC 1, BSC 40, BMT 10, VERO, W138, HeLa, Saos, C2C12, L cells, HT1080, HepG2, CHO. The selection of the mammalian species providing the cells is not a limitation of this invention; nor is the type of mammalian cell, i.e., fibroblast, hepatocyte, tumor cell, etc. Frequently used producer cells or HEK 293 cells and CHO cells. Preferentially, a producer cell should be free of potential adventitious viruses.

For purposes of this invention, “transfection” is used to refer to the uptake of nucleic acid compositions by a cell. A cell has been “transfected” when an exogenous nucleic acid composition has crossed the cell membrane. A number of transfection techniques are generally known in the art. Such techniques can be used to introduce one or more nucleic acid compositions, such as a plasmid vector and other nucleic acid molecules, into suitable host cells.

For purposes of this invention, by “vector”, “transfer vector”, “gene transfer vector” or “nucleic acid composition transfer vector” is meant any element, such as a plasmid, phage, transposon, cosmid, chromosome, virus, virus capsid, virion, etc., which is capable of transferring and/or transporting a nucleic acid composition to a host cell, into a host cell and/or to a specific location and/or compartment within a host cell. Thus, the term includes cloning and expression vehicles, as well as viral and non-viral vectors and potentially naked or complexed DNA. However, the term does not include cells that produce gene transfer vectors such as retroviral packaging cell lines.

For purposes of this invention, the term “control elements”, “regulatory sequences” or “regulatory control elements” refers collectively to promoter regions, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control elements need always be present as long as the selected coding sequence is capable of being replicated, transcribed and/or translated in an appropriate host cell. Sometimes, the entirety of control elements and coding sequence is referred to as “gene”; in other instances, “gene” only refers to the coding sequence. For purposes of this invention, “gene” refers to the entirety of control elements and coding sequence. Expression control elements include appropriate transcription initiation, termination, promoter and enhancer sequences, efficient RNA processing signals such as splicing and polyadenylation signals, sequences that stabilize cytoplasmic mRNA, sequences that enhance translation efficacy (i.e., Kozak consensus sequence), sequences that enhance protein stability, and when desired, sequences that enhance protein processing and/or secretion. A great number of expression control elements, e.g., native, constitutive, inducible and/or tissue specific, are known in the art and may be utilized to drive expression of the gene, depending upon the type of expression desired. For eukaryotic cells, expression control elements typically include a promoter, an enhancer, such as one derived from an immunoglobulin gene, SV40, cytomegalovirus, etc., a polyadenylation sequence, and may include splice donor and acceptor sites. The polyadenylation sequence generally is inserted following the transgene sequences and before the 3′ ITR sequence in rAAV vectors.

The regulatory sequences useful in the constructs of the present invention may also contain an intron, desirably located between the promoter/enhancer sequence and the gene. One possible intron sequence is derived from SV40, and is referred to as the SV40 T intron sequence. Another suitable regulatory sequence includes the woodchuck hepatitis virus post-transcriptional element. Still other methods may involve the use of a second internal promoter, an alternative splice signal, a co- or post-translational proteolytic cleavage strategy, among others which are known to those of skill in the art. Selection of these and other common vector and regulatory sequences are conventional, and many such sequences are available. See, e.g., Sambrook et al, and references cited therein at, for example, pages 3.18-3.26 and 16.17-16.27 and Ausubel et al., CURRENT PROTOCOLS IN MOLECULAR BIOLOGY, John Wiley & Sons, New York, 1989.

One of skill in the art may make a selection among these regulatory sequences without departing from the scope of this invention. Suitable promoter/enhancer sequences may be selected by one of skill in the art using the guidance provided by this application. Such selection is a routine matter and is not a limitation of the present invention.

For purposes of this invention, the term “promoter” means a regulatory sequence capable of binding RNA polymerase and/or a regulatory sequence sufficient to direct transcription. “Promoter” is also meant to encompass those promoter (or enhancer) elements for cell-type specific, tissue-specific and/or inducible (by external signals or agents) transcription; such elements may be located in the 5′ or 3′ regions of a native gene.

For purposes of this invention, the term “operative association” or “operative linkage” refers to an arrangement of elements or nucleic acid sequences wherein the components so described are configured so as to perform their intended function. Thus, (a) regulatory sequence(s) operably linked to a coding sequence is/are capable of effecting the expression of said coding sequence and is/are connected in such a way as to permit gene expression of the coding sequence when the appropriate molecules (e.g., transcriptional activator proteins) are bound to the regulatory sequence(s). The regulatory sequences need not be contiguous with the coding sequence, as long as they function to direct the expression thereof. Thus, for example, intervening untranslated yet transcribed sequences can be present between a promoter sequence and the coding sequence and the promoter sequence can still be considered “operably linked” to the coding sequence. “Operably linked” sequences include both expression control sequences that are contiguous with the coding sequences for the product of interest and expression control sequences that act in trans or at a distance to control the expression of the product of interest.

For purpose of this invention, the term “specific productivity” refers to the amount of the recombinant protein of interest produced by a single mammalian cell per day. For example a specific productivity of 20 pg/cell/day refers to the production of 20 pg of the recombinant protein of interest by a single mammalian cell within 24 hours.

For purpose of this invention, the term “batch yield” refers to the titer or concentration of the recombinant protein of interest produced by all of the mammalian cells in culture together. For secreted proteins, the “batch yield” refers to the concentration of the recombinant protein of interest in the culture medium with the recombinant protein of interest secreted into the medium by the mammalian cells present in the medium. For example, if a mammalian cell culture of 1 liter comprises 0.5 g of recombinant protein of interest in total, the batch yield is 500 mg/l. Thus, whereas the specific productivity refers to the production of recombinant protein by a single mammalian cell within one day, the batch yield refers to the cumulative production of recombinant protein by all the mammalian cells in the culture in respect to the culture volume.

For the purpose of this invention, the term “volumetric yield” refers to the absolute amount of recombinant protein produced by mammalian cells in a given volume. Thus, the volumetric yield equals the batch yield multiplied with the culture volume. For example, if the batch yield is 200 mg/l and the culture volume is 10 liters, then the volumetric yield equals 2,000 mg or 2 g (10 liters×200 mg/liter).

For the purpose of this invention, the term “PEI” refers to polyethyeleneimine in general and 25-kd linear polyethyleneimine (Polysciences, Eppelheim, Germany) in particular.

### (2) GENERAL METHODS

The practice of the present invention will employ, unless otherwise indicated, conventional methods of microbiology, molecular biology and recombinant DNA techniques within the skill of the art. Such techniques are explained fully in the literature; see, e.g., Sambrook, et al. Molecular Cloning: A Laboratory Manual (Current Edition); DNA Cloning: A Practical Approach, vol. I & II (D. Glover, ed.); Oligonucleotide Synthesis (N. Gait, ed., Current Edition); Nucleic Acid Hybridization (B. Hames & S. Higgins, eds., Current Edition); Transcription and Translation (B. Hames & S. Higgins, eds., Current Edition); CRC Handbook of Parvoviruses, vol. I & II (P. Tijessen, ed.); Fundamental Virology, 2nd Edition, vol. I & II (B. N. Fields and D. M. Knipe, eds.)

### EXAMPLE 1

### Influence of Cell Density During Transfection on Batch Yield

1 million (experiment 1.1; filter tube 1.1), 2 million (experiment 1.2; filter tube 1.2), 2.5 million (experiment 1.3; filter tube 1.3), 3 million (experiment 1.4; filter tube 1.4), 5 million (experiment 1.5; filter tube 1.5) and 10 million (experiment 1.6; filter tube 1.6) suspension-adapted HEK293E cells [1] were resuspended in 0.5 ml of Ex-Cell 293 HEK 293 serum-free medium with 4 mmol/l L-glutamine (Cat. No. 14571C-1000M; Lot No. 6A0093; SAFC Biosciences, Lenexa, Kans., USA, “Ex-Cell” medium; note: “Ex-Cell” medium—as used herein—refers to a medium comprising 4 mmol/l L-glutamine) in a 50-ml filter tube each (TPP AG, Trasadingen, Switzerland; Cat. No. 87050, Lot 20050174).

Then 25 μg plasmid DNA at a concentration of 1 μg/μl were added with the following composition

40% (8.75 μg) p-LC (SEQ ID NO: 1);
40% (8.75 μg) p-HC (SEQ ID NO: 3);
20% (2.5 μg) p-p21 (SEQ ID NO: 7).

Plasmid p-LC comprises the genetic information for the production of the light chain of an IgG antibody, plasmid p-HC comprises the genetic information for the production of the heavy chain of an IgG antibody, plasmid p-p21 comprises the genetic information for the production of the protein p21. The particular vector constructs used in the present invention can also be obtained from the inventors. The inventors can be contacted via e-mail at hildinger@gmx.net. Moreover, commercial services do exist that produce any desired nucleotide sequence even comprising several kilobase pairs including complete expression cassettes in plasmid backgrounds (e.g., Invitrogen, Carlsbad, USA; Geneart, Germany). Thus, providing the genetic sequence information of the plasmids should enable one of ordinary skill in the art to order the plasmids of the present invention at one of the commercial services listed.

After shortly mixing the DNA with the cells by gentle shaking, 50 μg of 25-kd linear polyethyleneimine (“PEI”; Polysciences, Eppelheim, Germany; [2]) were added at a concentration of 1 μg/μl to each of the tubes. After shortly mixing by gentle shaking, the filter tubes containing the cells together with the DNA and PEI were transferred into an orbital shaker (Kühner Shaker Cabinet ISF-4-W, “Kühner shaker”, Kühner AG, Birsfelden, Switzerland), and the cells were incubated for 4 hours at 37° C. in a 5% CO₂ atmosphere under shaking at 180 rpm. After that time, 4.5 ml of Ex-Cell medium were added to each of the filter tubes. Then, sodium butyrate was added to a final concentration of 3 mmol/l to each of the filter tubes. The cells then were returned into the Kühner shaker and incubated at 37° C. in a 5% CO₂ atmosphere under shaking at 180 rpm. Every seven days post transfectionem, cells were fed with 200 μl of a solution containing 400 g/l glucose and 200 mmol/l L-glutamine.

Each day, 25 μl of supernatant was removed from the cells in order to determine the antibody titer via ELISA. The ELISA was performed as published in prior art [3]. In short, Goat anti-human kappa light chain IgG (Biosource) was used for coating the ELISA-plates, and with AP-conjugated goat anti-human gamma chain IgG (Biosource) the synthesized IgG1 was detected. NPP was used as a substrate for the alkaline phosphatase. Absorption was measured at 405 nm against 490 nm using a microplate reader (SPECTRAmax™340; Molecular Devices, Palo Alto, Calif., USA).

The following results were obtained at day 7 after transfection:

| Experiment (filter tube) | Batch yield (mg/l) | Standard deviation (mg/l) |
|---|---|---|
| 1.1 (2e6 cells/ml; PCV 1) | 32 | 2.8 |
| 1.2 (4e6 cells/ml; PCV 2) | 129 | 11 |
| 1.3 (5e6 cells/ml; PCV 2.5) | 202 | 22 |
| 1.4 (6e6 cells/ml; PCV 3) | 241 | 13 |
| 1.5 (1e7 cells/ml; PCV 5) | 233 | 19 |
| 1.6 (2e7 cells/ml; PCV 10) | 268 | 16 |

As one can see, batch yields are higher with increasing cell density at the time of transfection. There seems to be a plateau at cell densities exceeding 6 million cells per ml (PCV 3) at the time of transfection.

Furthermore, the inventors performed RealTime PCR to quantify the amount of plasmids transferred into the cells in the context of experiment 1.1 and 1.6. For that purpose, 100 μl of cell culture was harvested at day 2 after transfection, the number of cells was quantified and the plasmid DNA was extracted using the plasmid DNA isolation kit of Roche Diagnostics according to manufacturer instructions. Then, RealTime PCR was performed to determine the number of plasmids. The results were as follows: There were approximately 100,000 plasmids per cell on average in experiment 1.6 (transfection at a cell density of 20 million cells per ml), but only approximately 15,000 plasmids per cell on average in experiment 1.1 (transfection at a cell density of 2 million cells per ml). These results clearly show the importance of transfecting at high cell densities to achieve optimal transfection efficacy.

### EXAMPLE 2

### Influence of the PEI-to-DNA Ratio on Batch Yield

In order to optimize the PEI-to-DNA ratio (on a weight/weight (w/w) basis), the inventors first determined the maximum amount of PEI that can be added to the cells without evoking toxicity. For that purpose, 100 million suspension-adapted HEK293E cells [1] were resuspended in 5 ml of Ex-Cell 293 HEK 293 serum-free medium with 4 mmol/l L-glutamine (Cat. No. 14571C-1000M; Lot No. 6A0093; SAFC Biosciences, Lenexa, Kans., USA, “Ex-Cell” medium) in a 50-ml filter tube (TPP AG, Trasadingen, Switzerland; Cat. No. 87050, Lot 20050174). Then, the cells were distributed into 10 50-ml filter tubes in aliquots of 0.5 ml each. Subsequently, the inventors added 10 μg of PEI (at a concentration of 1 μg/μl) to filter tube 1, 20 μg of PEI to filter tube 2, 30 μg of PEI to filter tube 3, etc.

After shortly mixing by gentle shaking, the filter tubes containing the cells with PEI were transferred into an orbital shaker (Kühner Shaker Cabinet ISF-4-W, “Kühner shaker”, Kühner AG, Birsfelden, Switzerland), and the cells were incubated for 4 hours at 37° C. in a 5% CO₂ atmosphere under shaking at 180 rpm. After that time, 4.5 ml of Ex-Cell medium were added. Then, sodium butyrate was added to a final concentration of 3 mmol/l. The cells then were returned into the Kühner shaker and incubated at 37° C. in a 5% CO₂ atmosphere under shaking at 180 rpm. Three days after transfection cell viability was analyzed microscopically using the trypan blue staining method.

The following results were obtained: Cell viability was in the range of 80% to 90% in filter tubes 1 to 5 (corresponding to the addition of a maximum of 50 μg of PEI). Viability started to drop with the addition of more than 50 μg of PEI—dropping to only ˜30% of viability with the addition of 100 μg of PEI (filter tube 10). Thus, 50 μg of PEI seems to be the optimal amount of PEI to be added to HEK293E cells.

In a next step, the optimal PEI-to-DNA ratio (w/w) was determined, leaving the absolute amount of PEI constant and only changing the amount of DNA added. For that purpose, 100 million suspension-adapted HEK293E cells [1] were resuspended in 5 ml of Ex-Cell 293 HEK 293 serum-free medium with 4 mmol/l L-glutamine (Cat. No. 14571C-1000M; Lot No. 6A0093; SAFC Biosciences, Lenexa, Kans., USA, “Ex-Cell” medium) in a 50-ml filter tube (TPP AG, Trasadingen, Switzerland; Cat. No. 87050, Lot 20050174). Then, the cells were distributed into 10 50-ml filter tubes in aliquots of 0.5 ml each.

Subsequently, the inventors added 0 μg of plasmid DNA to tube 1, 5 μg o of plasmid DNA to tube 2, 10 μg of plasmid DNA to tube 3, etc. The plasmid DNA (at a concentration of 1 μg/μl) had the following composition:

40% (8.75 μg) p-LC (SEQ ID NO: 1);
40% (8.75 μg) p-HC (SEQ ID NO: 3);
20% (2.5 μg) p-p21 (SEQ ID NO: 5).

Plasmid p-LC comprises the genetic information for the production of the light chain of an IgG antibody, plasmid p-HC comprises the genetic information for the production of the heavy chain of an IgG antibody, plasmid p-p21 comprises the genetic information for the production of the protein p21. The particular vector constructs used in the present invention can also be obtained from the inventors. The inventors can be contacted via e-mail at hildinger@gmx.net. Moreover, commercial services do exist that produce any desired nucleotide sequence even comprising several kilobase pairs including complete expression cassettes in plasmid backgrounds (e.g., Invitrogen, Carlsbad, USA; Geneart, Germany). Thus, providing the genetic sequence information of the plasmids should enable one of ordinary skill in the art to order the plasmids of the present invention at one of the commercial services listed.

After shortly mixing the DNA with the cells by gentle shaking, 50 μg of 25-kd linear polyethyleneimine (“PEI”; Polysciences, Eppelheim, Germany; [2]) were added at a concentration of 1 μg/μl to each of the 10 filter tubes. After shortly mixing by gentle shaking, the filter tubes containing the cells together with the DNA and PEI were transferred into an orbital shaker (Kühner Shaker Cabinet ISF-4-W, “Kühner shaker”, Kühner AG, Birsfelden, Switzerland), and the cells were incubated for 4 hours at 37° C. in a 5% CO₂ atmosphere under shaking at 180 rpm. After that time, 4.5 ml of Ex-Cell medium were added. Then, sodium butyrate was added to a final concentration of 3 mmol/l. The cells then were returned into the Kühner shaker and incubated at 37° C. in a 5% CO₂ atmosphere under shaking at 180 rpm. Every seven days post transfectionem, cells were fed with 200 μl of a solution containing 400 g/l glucose and 200 mmol/l L-glutamine.

Each day, 25 μl of supernatant was removed from the cells in order to determine the antibody titer via ELISA. The ELISA was performed as published in prior art [3]. In short, Goat anti-human kappa light chain IgG (Biosource) was used for coating the ELISA-plates, and with AP-conjugated goat anti-human gamma chain IgG (Biosource) the synthesized IgG1 was detected. NPP was used as a substrate for the alkaline phosphatase. Absorption was measured at 405 nm against 490 nm using a microplate reader (SPECTRAmax™340; Molecular Devices, Palo Alto, Calif., USA).

The following results were obtained at day 6:

| Filter tube (experiment) | Batch yield (mg/l) |
|---|---|
| 2.1 (no DNA added) | 0 |
| 2.2 (5 μg DNA) | 42 |
| 2.3 (10 μg DNA) | 96 |
| 2.4 (15 μg DNA) | 150 |
| 2.5 (20 μg DNA) | 183 |
| 2.6 (25 μg DNA) | 227 |
| 2.7 (30 μg DNA) | 219 |
| 2.8 (35 μg DNA) | 194 |
| 2.9 (40 μg DNA) | 170 |
| 2.10 (45 μg DNA) | 143 |

As one can see, yields reach a plateau at a PEI-to-DNA ratio of 2:1 (w/w), i.e., in the experimental settings chosen 50 μg PEI in combination with 25 μg of DNA yielded the best results for the transfection of 10 million HEK 293E cells in 0.5 ml of Ex-Cell medium.

The optimization procedure as outlined in the previous two paragraphs can be performed with any cell line of interest without undue effort. To summarize: The artist first determines the PEI toxicity for the cell line in question and then uses the maximum amount of PEI that can be tolerated for the cell line in question. Then, in a second step, the artist uses a fixed amount of PEI (which s/he determined in the first experiment) and combines it with increasing amounts of DNA. Using this simple two-step approach, the optimal PEI-to-DNA ratio can be determined for any cell line of interest without undue effort.

### EXAMPLE 3

### Influence of Sequencing of PEI-DNA-Cell Mixture

In this example, the inventors investigated the impact of either adding the DNA to the cells and then the PEI, or adding the PEI to the cells first and then adding the DNA, or combining PEI with DNA first, prior to adding PEI:DNA complexes to the cells.

The experiment was performed as described in the “Preferred Embodiment” with the exception that in one instance, the DNA was added first to the cells followed by the addition of PEI (as outlined in the preferred embodiment; experiment 3.1), in another instance, the PEI was added first to the cells followed by the addition of the DNA (experiment 3.2), and yet in another instance, the PEI-DNA-complexes were preformed prior to adding them to the DNA (as described in prior art [2]).

The following results were obtained for day 7:

| | | Standard |
|---|---|---|
| Experiment | Batch yield (mg/l) | deviation (mg/l) |
| 3.1 (DNA, then PEI) | 413 | 37 |
| 3.2 (PEI, then DNA) | 378 | 27 |
| 3.3 (DNA + PEI pre-complex) | 285 | 24 |

As one can see, adding the DNA first and then the PEI, is—on average—slightly better than adding first the PEI and then the DNA—although the difference is statistically not significant. On the other hand, a pre-complex formation of DNA and PEI is significantly worse than the other two options.

### EXAMPLE 4

### Influence of Medium Composition and Serum on Batch Yield

In order to investigate the influence of medium composition and serum on batch yield, the inventors performed the following experiment:

30 million suspension-adapted HEK293E cells [1] were resuspended in 1.5 ml of Ex-Cell 293 HEK 293 serum-free medium with 4 mmol/l L-glutamine (Cat. No. 14571C-1000M; Lot No. 6A0093; SAFC Biosciences, Lenexa, Kans., USA, “Ex-Cell” medium) in a 50-ml filter tube (TPP AG, Trasadingen, Switzerland; Cat. No. 87050, Lot 20050174).

Then 75 μg plasmid DNA at a concentration of 1 μg/μl were added with the following composition

40% (30 μg) p-LC (SEQ ID NO: 1);
40% (30 μg) p-HC (SEQ ID NO: 3);
10% (7.5 μg) p-p18 (SEQ ID NO: 5);
10% (7.5 μg) p-p21 (SEQ ID NO: 7);

Plasmid p-LC comprises the genetic information for the production of the light chain of an IgG antibody, plasmid p-HC comprises the genetic information for the production of the heavy chain of an IgG antibody, plasmid p-p18 comprises the genetic information for the production of the protein p18, plasmid p-p21 comprises the genetic information for the production of the protein p21. The particular vector constructs used in the present invention can also be obtained from the inventors. The inventors can be contacted via e-mail at hildinger@gmx.net. Moreover, commercial services do exist that produce any desired nucleotide sequence even comprising several kilobase pairs including complete expression cassettes in plasmid backgrounds (e.g., Invitrogen, Carlsbad, USA; Geneart, Germany). Thus, providing the genetic sequence information of the plasmids should enable one of ordinary skill in the art to order the plasmids of the present invention at one of the commercial services listed.

After shortly mixing the DNA with the cells by gentle shaking, 150 μg of 25-kd linear polyethyleneimine (“PEI”; Polysciences, Eppelheim, Germany; [2]) were added at a concentration of 1 μg/μl. After shortly mixing by gentle shaking, the filter tube containing the cells together with the DNA and PEI was transferred into an orbital shaker (Kühner Shaker Cabinet ISF-4-W, “Kühner shaker”, Kühner AG, Birsfelden, Switzerland), and the cells were incubated for 4 hours at 37° C. in a 5% CO₂ atmosphere under shaking at 180 rpm.

After that time, the 1.5 ml of cells were distributed into 3 new filter tubes with 0.5 ml each. Then, 4.5 ml of Ex-Cell medium were added to filter tube 1 (experiment 4.1), 4.5 ml of Ex-Cell medium with 10% Fetal Calf Serum were added to filter tube 2 (experiment 4.2) and 4.5 ml of FreeStyle medium (Cat. No. 12338, Invitrogen, Carlsbad, USA, FreeStyle™ 293 Expression Medium) were added to filter tube 3 (experiment 4.3). Then, sodium butyrate was added to each tube to a final concentration of 3 mmol/l. The cells then were returned into the Kühner shaker and incubated at 37° C. in a 5% CO₂ atmosphere under shaking at 180 rpm. Every seven days post transfectionem, cells were fed with 200 μl of a solution containing 400 g/l glucose and 200 mmol/l L-glutamine.

Each day, 25 μl of supernatant was removed from the cells in order to determine the antibody titer via ELISA. The ELISA was performed as published in prior art [3]. In short, Goat anti-human kappa light chain IgG (Biosource) was used for coating the ELISA-plates, and with AP-conjugated goat anti-human gamma chain IgG (Biosource) the synthesized IgG1 was detected. NPP was used as a substrate for the alkaline phosphatase. Absorption was measured at 405 nm against 490 nm using a microplate reader (SPECTRAmax™340; Molecular Devices, Palo Alto, Calif., USA).

The following results were obtained at day 7:

| Experiment (filter tube) | Batch yield (mg/l) | Standard deviation (mg/l) |
|---|---|---|
| 4.1 (Ex-Cell medium) | 318 | 28 |
| 4.2 (Ex-Cell medium with | 309 | 34 |
| 10% FCS) | | |
| 4.3 (FreeStyle medium) | 362 | 31 |

As one can see, the addition of serum has no effect on antibody titers. (Yet, it should be noted that serum had a positive effect on GFP expression in a similar experiment; data not shown). On average, FreeStyle medium performed slightly better than Ex-Cell medium, but not to the level of statistical significance within one standard deviation.

### EXAMPLE 5

### Influence of Cell Density after Transfection on Batch Yield

To analyze the influence of cell density after transfection on batch yield, the inventors performed the following experiment:

200 million suspension-adapted HEK293E cells [1] were resuspended in 10 ml of Ex-Cell 293 HEK 293 serum-free medium with 4 mmol/l L-glutamine (Cat. No. 14571C-1000M; Lot No. 6A0093; SAFC Biosciences, Lenexa, Kans., USA, “Ex-Cell” medium) in a 50-ml filter tube (TPP AG, Trasadingen, Switzerland; Cat. No. 87050, Lot 20050174).

Then 500 μg plasmid DNA at a concentration of 1 μg/μl were added with the following composition

40% (200 μg) p-LC (SEQ ID NO: 1);
40% (200 μg) p-HC (SEQ ID NO: 3);
10% (50 μg) p-p18 (SEQ ID NO: 5);
10% (50 μg) p-p21 (SEQ ID NO: 7);

Plasmid p-LC comprises the genetic information for the production of the light chain of an IgG antibody, plasmid p-HC comprises the genetic information for the production of the heavy chain of an IgG antibody, plasmid p-p18 comprises the genetic information for the production of the protein p18, plasmid p-p21 comprises the genetic information for the production of the protein p21. The particular vector constructs used in the present invention can also be obtained from the inventors. The inventors can be contacted via e-mail at hildinger@gmx.net. Moreover, commercial services do exist that produce any desired nucleotide sequence even comprising several kilobase pairs including complete expression cassettes in plasmid backgrounds (e.g., Invitrogen, Carlsbad, USA; Geneart, Germany). Thus, providing the genetic sequence information of the plasmids should enable one of ordinary skill in the art to order the plasmids of the present invention at one of the commercial services listed.

After shortly mixing the DNA with the cells by gentle shaking, 1,000 μg of 25-kd linear polyethyleneimine (“PEI”; Polysciences, Eppelheim, Germany; [2]) were added at a concentration of 1 μg/μl. After shortly mixing by gentle shaking, the filter tube containing the cells together with the DNA and PEI was transferred into an orbital shaker (Kühner Shaker Cabinet ISF-4-W, “Kühner shaker”, Kühner AG, Birsfelden, Switzerland), and the cells were incubated for 4 hours at 37° C. in a 5% CO₂ atmosphere under shaking at 180 rpm.

After that time, 0.25 ml of transfected cells were added to a filter tube with 4.75 ml of Ex-Cell medium (experiment 5.1), 0.5 ml of transfected cells were added to a filter tube with 4.5 ml of Ex-Cell medium (experiment 5.2), 0.75 ml of transfected cells were added to a filter tube with 4.25 ml of Ex-Cell medium (experiment 5.3), 1 ml of transfected cells were added to a filter tube with 4 ml of Ex-Cell medium (experiment 5.4), 1.5 ml of transfected cells were added to a filter tube with 3.5 ml of Ex-Cell medium (experiment 5.5), 2 ml of transfected cells were added to a filter tube with to 3 ml of Ex-Cell medium (experiment 5.6).

Then, sodium butyrate was added to a final concentration of 3 mmol/l into each of the filter tubes. The cells then were returned into the Kühner shaker and incubated at 37° C. in a 5% CO₂ atmosphere under shaking at 180 rpm. Every seven days post transfectionem, cells were fed with 200 μl of a solution containing 400 g/l glucose and 200 mmol/l L-glutamine.

Each day, 25 μl of supernatant was removed from the cells in order to determine the antibody titer via ELISA. The ELISA was performed as published in prior art [3]. In short, Goat anti-human kappa light chain IgG (Biosource) was used for coating the ELISA-plates, and with AP-conjugated goat anti-human gamma chain IgG (Biosource) the synthesized IgG1 was detected. NPP was used as a substrate for the alkaline phosphatase. Absorption was measured at 405 nm against 490 nm using a microplate reader (SPECTRAmax™340; Molecular Devices, Palo Alto, Calif., USA).

The following results were obtained for day 8 after transfection:

| Experiment | Batch yield (mg/l) | Standard deviation (mg/l) |
|---|---|---|
| 5.1 (1e6 cells/ml; PCV 0.5) | 152 | 18 |
| 5.2 (2e6 cells/ml; PCV 1) | 344 | 24 |
| 5.3 (3e6 cells/ml; PCV 1.5) | 367 | 31 |
| 5.4 (4e6 cells/ml; PCV 2) | 391 | 34 |
| 5.5 (5e6 cells/ml; PCV 2.5) | 208 | 17 |
| 5.6 (6e6 cells/ml; PCV 3) | 173 | 8 |

As one can see, a cell density of 2 million cells/ml is preferable over a cell density of 1 million cells/ml. Whereas titers further increase with increasing cell density of up to 4 million cells/ml, the yield increase is getting smaller. Above a cell density of 4 million cells/ml, yields start to decrease.

### EXAMPLE 6

### Influence of Sodium Butyrate on Batch Yield

To analyze the influence of sodium butyrate addition on batch yield, the inventors performed an experiment as outlined in “Preferred Embodiment” with the difference of adding 1 mmol/l sodium butyrate (experiment 6.1), 2 mmol/l sodium butyrate (experiment 6.2), 3 mmol/l sodium butyrate (experiment 6.3; “Preferred Embodiment”), 6 mmol/l sodium butyrate (experiment 6.4) and 12 mmol/l sodium butyrate (experiment 6.5) or no sodium butyrate (experiment 6.6) with sodium butyrate referred to as “NaBut”.

The results at day 14 were as follows:

| Experiment | Batch yield (mg/l) | Standard deviation (mg/l) |
|---|---|---|
| 6.1 (1 mmol/l NaBut) | 419 | 53 |
| 6.2 (2 mmol/l NaBut) | 632 | 67 |
| 6.3 (3 mmol/l NaBut) | 673 | 58 |
| 6.4 (6 mmol/NaBut) | 447 | 36 |
| 6.5 (12 mmol/l NaBut) | 288 | 45 |
| 6.6 (no NaBut added) | 68 | 2.9 |

As one can see, an optimum of sodium butyrate addition is achieved at around 3 mmol/l, where addition of 2 mmol/l is statistically not different from the addition of 3 mmol/l. Yet, without the addition of sodium butyrate, batch yields are extremely low. Furthermore, when performing the transfection without co-transfecting p18 and p21, yields in the absence of sodium butyrate are even lower than in experiment 6.6—being in the range of 30 mg/l to 40 mg/l of average maximum batch yields.

Furthermore, it should be noted that the addition of sodium butyrate can also occur at a later time point than directly after transfection. Here, the critical aspect is to add the sodium butyrate prior to the cells reaching a PCV of 2.2. Addition of sodium butyrate after that cell density has been reached will not prolong cell survival as it takes approximately 1 day for the sodium butyrate to growth arrest the cells.

### EXAMPLE 7

### Influence of 2-Aminopurine on Batch Yield

In order to investigate the influence of the addition of 2-aminopurine on batch yield, the inventors performed the following experiment:

30 million suspension-adapted HEK293E cells [1] were resuspended in 1.5 ml of Ex-Cell 293 HEK 293 serum-free medium with 4 mmol/l L-glutamine (Cat. No. 14571C-1000M; Lot No. 6A0093; SAFC Biosciences, Lenexa, Kans., USA, “Ex-Cell” medium) in a 50-ml filter tube (TPP AG, Trasadingen, Switzerland; Cat. No. 87050, Lot 20050174).

Then 75 μg plasmid DNA at a concentration of 1 μg/μl were added with the following composition

40% (30 μg) p-LC (SEQ ID NO: 1);
40% (30 μg) p-HC (SEQ ID NO: 3);
10% (7.5 μg) p-p18 (SEQ ID NO: 5);
10% (7.5 μg) p-p21 (SEQ ID NO: 7);

Plasmid p-LC comprises the genetic information for the production of the light chain of an IgG antibody, plasmid p-HC comprises the genetic information for the production of the heavy chain of an IgG antibody, plasmid p-p18 comprises the genetic information for the production of the protein p18, plasmid p-p21 comprises the genetic information for the production of the protein p21. The particular vector constructs used in the present invention can also be obtained from the inventors. The inventors can be contacted via e-mail at hildinger@gmx.net. Moreover, commercial services do exist that produce any desired nucleotide sequence even comprising several kilobase pairs including complete expression cassettes in plasmid backgrounds (e.g., Invitrogen, Carlsbad, USA; Geneart, Germany). Thus, providing the genetic sequence information of the plasmids should enable one of ordinary skill in the art to order the plasmids of the present invention at one of the commercial services listed.

After shortly mixing the DNA with the cells by gentle shaking, 150 μg of 25-kd linear polyethyleneimine (“PEI”; Polysciences, Eppelheim, Germany; [2]) were added at a concentration of 1 μg/μl. After shortly mixing by gentle shaking, the filter tube containing the cells together with the DNA and PEI was transferred into an orbital shaker (Kühner Shaker Cabinet ISF-4-W, “Kühner shaker”, Kühner AG, Birsfelden, Switzerland), and the cells were incubated for 4 hours at 37° C. in a 5% CO₂ atmosphere under shaking at 180 rpm.

After that time, 13.5 ml of Ex-Cell medium comprising 1× antibiotic, antimycotic solution (Sigma Aldrich; A5955; Lot 103K2393) was added to the cells as well as sodium butyrate to a final concentration of 3 mmol/l. Then, the content of the filter tube was distributed equally into three new filter tubes with 5 ml of cell suspension each. The inventors added then 5 mmol/l 2-aminopurine (final concentration) to filter tube 2 (experiment 7.2) and 10 mmol/l 2-aminopurine (final concentration) to filter tube 3 (experiment 7.3). The cells then were returned into the Kühner shaker and incubated at 37° C. in a 5% CO₂ atmosphere under shaking at 180 rpm. Every seven days post transfectionem, cells were fed with 200 μl of a solution containing 400 g/l glucose and 200 mmol/l L-glutamine.

Each day, 25 μl of supernatant was removed from the cells in order to determine the antibody titer via ELISA. The ELISA was performed as published in prior art [3]. In short, Goat anti-human kappa light chain IgG (Biosource) was used for coating the ELISA-plates, and with AP-conjugated goat anti-human gamma chain IgG (Biosource) the synthesized IgG1 was detected. NPP was used as a substrate for the alkaline phosphatase. Absorption was measured at 405 nm against 490 nm using a microplate reader (SPECTRAmax™340; Molecular Devices, Palo Alto, Calif., USA).

The following results were obtained at day 18:

| Experiment | Batch yield (mg/l) | Standard deviation (mg/l) |
|---|---|---|
| 7.1 (control) | 553 | 43 |
| 7.2 (5 mmol/l 2- | 628 | 72 |
| aminopurine) | | |
| 7.3 (10 mmol/l 2- | 708 | 66 |
| aminopurine) | | |

As one can see, the addition of 2-aminopurine significantly increases batch yield. The difference between adding 5 mmol/l and 10 mmol/l 2-aminopurine was not statistically significant, yet, on average, adding 10 mmol/l was slightly better. However, given the current cost of 2-aminopurine, it will be challenging to use this protocol for larger scale recombinant protein production.

### EXAMPLE 8

### Influence of Temperature on Batch Yield

40 million suspension-adapted CHO DG44 cells were resuspended in 2 ml of ProCHO5 serum-free medium (BioWhittaker; Cambrex; Cat. No. BE12-766Q; Lot No. 5MB0106) with 4 mmol/l L-glutamine and nucleosides (HT) in a 50-ml filter tube (TPP AG, Trasadingen, Switzerland; Cat. No. 87050, Lot 20050174).

Then 100 μg plasmid DNA at a concentration of 1 μg/μl were added with the following composition:

40% (40 μg) p-LC (SEQ ID NO: 1);
40% (40 μg) p-HC (SEQ ID NO: 3);
10% (10 μg) p-p18 (SEQ ID NO: 5);
10% (10 μg) p-p21 (SEQ ID NO: 7);

Plasmid p-LC comprises the genetic information for the production of the light chain of an IgG antibody, plasmid p-HC comprises the genetic information for the production of the heavy chain of an IgG antibody, plasmid p-p18 comprises the genetic information for the production of the protein p18, plasmid p-p21 comprises the genetic information for the production of the protein p21. The particular vector constructs used in the present invention can also be obtained from the inventors. The inventors can be contacted via e-mail at hildinger@gmx.net. Moreover, commercial services do exist that produce any desired nucleotide sequence even comprising several kilobase pairs including complete expression cassettes in plasmid backgrounds (e.g., Invitrogen, Carlsbad, USA; Geneart, Germany). Thus, providing the genetic sequence information of the plasmids should enable one of ordinary skill in the art to order the plasmids of the present invention at one of the commercial services listed.

After shortly mixing the DNA with the cells by gentle shaking, 200 μg of 25-kd linear polyethyleneimine (“PEI”; Polysciences, Eppelheim, Germany; [2]) were added at a concentration of 1 μg/μl. After shortly mixing by gentle shaking, the filter tube containing the cells together with the DNA and PEI was transferred into an orbital shaker (Kühner Shaker Cabinet ISF-4-W, “Kühner shaker”, Kühner AG, Birsfelden, Switzerland), and the cells were incubated for 4 hours at 37° C. in a 5% CO₂ atmosphere under shaking at 180 rpm. After that time, 18 ml of ProCHO5 medium were added. The cells were then equally distributed into 4 new 50-ml filter tubes (i.e., 5 ml of cell suspension in each tube) and treated as follows:

Tubes 8.1 and 8.3: Addition of sodium butyrate to a final concentration of 2 mmol/l

Tubes 8.2 and 8.4: No addition of sodium butyrate.

Tubes 8.1 and 8.2 were then returned into a Kühner shaker and incubated at 37° C. in a 5% CO₂ atmosphere under shaking at 180 rpm. Tubes 8.3 and 8.4 were then returned into a Kühner shaker and incubated at 31° C. in a 5% CO₂ atmosphere under shaking at 180 rpm.

Every seven days post transfectionem, cells were fed with 200 μl of a solution containing 400 g/l glucose and 200 mmol/l L-glutamine.

Each day, 25 μl of supernatant was removed from the cells in order to determine the antibody titer via ELISA. The ELISA was performed as published in prior art [3]. In short, Goat anti-human kappa light chain IgG (Biosource) was used for coating the ELISA-plates, and with AP-conjugated goat anti-human gamma chain IgG (Biosource) the synthesized IgG1 was detected. NPP was used as a substrate for the alkaline phosphatase. Absorption was measured at 405 nm against 490 nm using a microplate reader (SPECTRAmax™340; Molecular Devices, Palo Alto, Calif., USA).

The following results were obtained as peak values during the 18 day cultivation period:

| | Maximum batch | Standard deviation |
|---|---|---|
| Experiment | yield (mg/l) | (mg/l) |
| Tube 8.1 (37° C.; NaBut) | 218 | 17 |
| Tube 8.2 (37° C.; no NaBut) | 58 | 4.8 |
| Tube 8.3 (31° C.; NaBut) | 231 | 19 |
| Tube 8.4 (31° C.; no NaBut) | 225 | 21 |

As one can see, batch yields exceeding 200 mg/l could be obtained with and without addition of sodium butyrate and at 31° C. and 37° C., respectively. Yet, without the addition of sodium butyrate or without temperature shifting, yields are extremely low. Moreover, the combination of temperature shift and sodium butyrate did not act synergistically on batch yields.

### EXAMPLE 9

### Influence of Co-Transfection of p18 and p21 on Batch Yield

40 million suspension-adapted HEK293E cells [1] were resuspended in 2 ml of Ex-Cell 293 HEK 293 serum-free medium with 4 mmol/l L-glutamine (Cat. No. 14571C-1000M; Lot No. 6A0093; SAFC Biosciences, Lenexa, Kans., USA, “Ex-Cell” medium) in a 50-ml filter tube (TPP AG, Trasadingen, Switzerland; Cat. No. 87050, Lot 20050174).

Then, the cells were distributed equally into four new 50 ml filter tubes, i.e., each tube received 0.5 ml of cells. Then, the following amounts of DNA were added to the tubes (referred to as tubes 9.1; 9.2; 9.3; 9.4) at a concentration of 1 μg/μl:

40% (8.75 μg) p-LC (SEQ ID NO: 1): Tubes 9.1; 9.2; 9.3; 9.4
40% (8.75 μg) p-HC (SEQ ID NO: 3): Tubes 9.1; 9.2; 9.3; 9.4
10% (2.5 μg) p-p18 (SEQ ID NO: 5): Tubes 9.2; 9.4
10% (2.5 μg) p-p21 (SEQ ID NO: 7): Tubes 9.3; 9.4

Plasmid p-LC comprises the genetic information for the production of the light chain of an IgG antibody, plasmid p-HC comprises the genetic information for the production of the heavy chain of an IgG antibody, plasmid p-p18 comprises the genetic information for the production of the protein p18, plasmid p-p21 comprises the genetic information for the production of the protein p21. The particular vector constructs used in the present invention can also be obtained from the inventors. The inventors can be contacted via e-mail at hildinger@gmx.net. Moreover, commercial services do exist that produce any desired nucleotide sequence even comprising several kilobase pairs including complete expression cassettes in plasmid backgrounds (e.g., Invitrogen, Carlsbad, USA; Geneart, Germany). Thus, providing the genetic sequence information of the plasmids should enable one of ordinary skill in the art to order the plasmids of the present invention at one of the commercial services listed.

After shortly mixing the DNA with the cells by gentle shaking, 50 μg of 25-kd linear polyethyleneimine (“PEI”; Polysciences, Eppelheim, Germany; [2]) were added at a concentration of 1 μg/μl to each of the 4 tubes. After shortly mixing by gentle shaking, the four filter tubes containing the cells together with the DNA and PEI were transferred into an orbital shaker (Kühner Shaker Cabinet ISF-4-W, “Kühner shaker”, Kühner AG, Birsfelden, Switzerland), and the cells were incubated for 4 hours at 37° C. in a 5% CO₂ atmosphere under shaking at 180 rpm. After that time, 4.5 ml of Ex-Cell medium were added to each of the four tubes. Then, sodium butyrate was added to a final concentration of 3 mmol/l. The cells then were returned into the Kühner shaker and incubated at 37° C. in a 5% CO₂ atmosphere under shaking at 180 rpm. Every seven days post transfectionem, cells were fed in each tube with 200 μl of a solution containing 400 g/l glucose and 200 mmol/l L-glutamine.

Each day, 25 μl of supernatant was removed from the cells in order to determine the antibody titer via ELISA. The ELISA was performed as published in prior art [3]. In short, Goat anti-human kappa light chain IgG (Biosource) was used for coating the ELISA-plates, and with AP-conjugated goat anti-human gamma chain IgG (Biosource) the synthesized IgG1 was detected. NPP was used as a substrate for the alkaline phosphatase. Absorption was measured at 405 nm against 490 nm using a microplate reader (SPECTRAmax™340; Molecular Devices, Palo Alto, Calif., USA).

The following results were obtained at day 8 after transfection:

| Experiment | Batch yield (mg/l) | Standard deviation (mg/l) |
|---|---|---|
| Tube 9.1 (control) | 238 | 23 |
| Tube 9.2 (p18 only) | 305 | 28 |
| Tube 9.3 (p21 only) | 324 | 27 |
| Tube 9.4 (p18 and p21) | 387 | 22 |

As one can see, the addition of p18 and p21 alone significantly increases titers. In addition, the combination of p18 and p21 further increases titers. The investigators also analyzed the impact of using 20% p21 or 20% p18 instead of the combination of p21 and p18; yet, the combination of 10% p18 and 10% p21 on average achieved higher batch yields compared to the use of either 20% p21 or 20% p18.

### EXAMPLE 10

### Expression of a Soluble Fc-Tagged TNF-Alpha Receptor

10 million suspension-adapted HEK293E cells [1] were resuspended in 0.5 ml of Ex-Cell 293 HEK 293 serum-free medium with 4 mmol/l L-glutamine (Cat. No. 14571C-1000M; Lot No. 6A0093; SAFC Biosciences, Lenexa, Kans., USA, “Ex-Cell” medium) in a 50-ml filter tube (TPP AG, Trasadingen, Switzerland; Cat. No. 87050, Lot 20050174).

Then 25 μg plasmid DNA at a concentration of 1 μg/μl were added with the following composition:

80% (20 μg) p-Enbrel (SEQ ID NO: 11);
10% (2.5 μg) p-p18 (SEQ ID NO: 5);
10% (2.5 μg) p-p21 (SEQ ID NO: 7);

Plasmid p-Enbrel comprises the genetic information for the production of an Fc-tagged, soluble TNF-alpha receptor, plasmid p-p18 comprises the genetic information for the production of the protein p18, plasmid p-p21 comprises the genetic information for the production of the protein p21. The particular vector constructs used in the present invention can also be obtained from the inventors. The inventors can be contacted via e-mail at hildinger@gmx.net. Moreover, commercial services do exist that produce any desired nucleotide sequence even comprising several kilobase pairs including complete expression cassettes in plasmid backgrounds (e.g., Invitrogen, Carlsbad, USA; Geneart, Germany). Thus, providing the genetic sequence information of the plasmids should enable one of ordinary skill in the art to order the plasmids of the present invention at one of the commercial services listed.

After shortly mixing the DNA with the cells by gentle shaking, 50 μg of 25-kd linear polyethyleneimine (“PEI”; Polysciences, Eppelheim, Germany; [2]) were added at a concentration of 1 μg/μl. After shortly mixing by gentle shaking, the filter tube containing the cells together with the DNA and PEI was transferred into an orbital shaker (Kühner Shaker Cabinet ISF-4-W, “Kühner shaker”, Kühner AG, Birsfelden, Switzerland), and the cells were incubated for 4 hours at 37° C. in a 5% CO₂ atmosphere under shaking at 180 rpm. After that time, 9 ml of Ex-Cell medium were added. Then, sodium butyrate was added to a final concentration of 3 mmol/l. The cells then were returned into the Kühner shaker and incubated at 37° C. in a 5% CO₂ atmosphere under shaking at 180 rpm. Every seven days post transfectionem, cells were fed with 200 μl of a solution containing 400 g/l glucose and 200 mmol/l L-glutamine.

Each day, 25 μl of supernatant was removed from the cells in order to determine the titer via ELISA. The ELISA was performed as published in prior art [3] with the exception of using an anti-human-Fc antibody as coating antibody.

The following results were obtained:

| Day after transfection | Titer (mg/l) |
|---|---|
| 0 | — |
| 1 | 28.5 |
| 2 | 69.9 |
| 3 | 144.0 |
| 4 | 219.7 |
| 5 | 291.9 |
| 6 | 365.7 |
| 7 | 420.2 |
| 8 | 485.6 |
| 9 | 539.8 |
| 10 | 582.1 |
| 11 | 617.3 |
| 12 | 645.6 |
| 13 | 670.6 |
| 14 | 684.8 |
| 15 | 697.7 |
| 16 | 707.6 |
| 17 | 712.8 |
| 18 | 714.2 |

As one can see, yields beyond 500 mg/l could be achieved. It has to be noted that the cell density after transfection in that experiment was 1 million cells/ml, not 2 million cells/ml. Assuming a cell density of 2 million cells/ml instead of 1 million cells/ml, the inventors should achieve a batch yield exceeding 700 mg/l and even 1 g/l.

### EXAMPLE 11

### 2 Gram of Volumetric Yield in a 20-Liter Non-Instrumented, Shaken “Bioreactor” with 10 Liter of Culture Volume

20 billion suspension-adapted HEK293E cells [1] were resuspended in 1 liter of Ex-Cell 293 HEK 293 serum-free medium with 4 mmol/l L-glutamine (Cat. No. 14571C-1000M; Lot No. 6A0093; SAFC Biosciences, Lenexa, Kans., USA, “Ex-Cell” medium) in a 5 liter round-shaped Schott Duran glass bottle (Cat. No. 21 801 73; Schott AG, Mainz, Germany).

Then 50 mg plasmid DNA at a concentration of 1 μg/μl were added with the following composition

40% (20 mg) p-LC (SEQ ID NO: 1);
40% (20 mg) p-HC (SEQ ID NO: 3);
10% (2.5 μg) p-p18 (SEQ ID NO: 5);
10% (2.5 μg) p-p21 (SEQ ID NO: 7).

Plasmid p-LC comprises the genetic information for the production of the light chain of an IgG antibody, plasmid p-HC comprises the genetic information for the production of the heavy chain of an IgG antibody, plasmid p-p18 comprises the genetic information for the production of the protein p18, plasmid p-p21 comprises the genetic information for the production of the protein p21. The particular vector constructs used in the present invention can also be obtained from the inventors. The inventors can be contacted via e-mail at hildinger@gmx.net. Moreover, commercial services do exist that produce any desired nucleotide sequence even comprising several kilobase pairs including complete expression cassettes in plasmid backgrounds (e.g., Invitrogen, Carlsbad, USA; Geneart, Germany). Thus, providing the genetic sequence information of the plasmids should enable one of ordinary skill in the art to order the plasmids of the present invention at one of the commercial services listed.

After shortly mixing the DNA with the cells by gentle shaking, 100 mg of 25-kd linear polyethyleneimine (“PEI”; Polysciences, Eppelheim, Germany; [2]) were added at a concentration of 1 mg/ml. After shortly mixing by gentle shaking, the glass bottle containing the cells together with the DNA and PEI was transferred into an orbital shaker (Kühner Shaker Cabinet ISF-4-W, “Kühner shaker”, Kühner AG, Birsfelden, Switzerland), and the cells were incubated for 4 hours at 37° C. in a 5% CO₂ atmosphere under shaking at 90 rpm. After that time, the cells were transferred into a 20 liter Nalgene bottle (Nalgene 20 L Single-Use Sterile Carboy, HDPE, 83B Screw Cap) with 9 liters of 37° C. pre-warmed Ex-Cell medium. Then, sodium butyrate was added to a final concentration of 3 mmol/l. The cells then were returned into the Kühner shaker and incubated at 37° C. in a 5% CO₂ atmosphere under shaking at 80 rpm. Every seven days post transfectionem, cells were fed with 400 ml of a solution containing 400 g/l glucose and 200 mmol/l L-glutamine.

Each day, 50 ml of supernatant was removed from the cells in order to determine the antibody titer via ELISA. The ELISA was performed as published in prior art [3]. In short, Goat anti-human kappa light chain IgG (Biosource) was used for coating the ELISA-plates, and with AP-conjugated goat anti-human gamma chain IgG (Biosource) the synthesized IgG1 was detected. NPP was used as a substrate for the alkaline phosphatase. Absorption was measured at 405 nm against 490 nm using a microplate reader (SPECTRAmax™340; Molecular Devices, Palo Alto, Calif., USA).

At day 7, a batch yield of 230 mg/l was measured, resulting in a volumetric yield of 2.3 g.

### EXAMPLE 12

### Influence of the Timing of Dilution after Transfection on Batch Yield

In a further experiment, the inventors investigated the optimal timing of dilution after transfection. For that purpose, the inventors transfected cells as described in “Preferred Embodiment” with the exception of diluting the cells after 1 hour, 2 hours, 3 hours, 4 hours, 5 hours, 6 hours and 8 hours after transfection. In summary, the optimal time point for dilution was at 3 to 4 hours (with 4 hours on average resulting in higher yields compared to dilution after 3 hours, but not to the level of statistical significance), whereas yields were only approximately half when diluting after 1 hour, and decreased at about 20% with each hour of dilution performed after the 4 hour time point.

### Preferred Embodiment

10 million suspension-adapted HEK293E cells [1] were resuspended in 0.5 ml of Ex-Cell 293 HEK 293 serum-free medium with 4 mmol/l L-glutamine (Cat. No. 14571C-1000M; Lot No. 6A0093; SAFC Biosciences, Lenexa, Kans., USA, “Ex-Cell” medium) in a 50-ml filter tube (TPP AG, Trasadingen, Switzerland; Cat. No. 87050, Lot 20050174). In order to determine the cell density, the packed cell volume (PCV) was determined as described in prior art [4]—assuming a PCV of 0.5 corresponds to 1 million HEK293E cells per ml.

Then 25 μg plasmid DNA at a concentration of 1 μg/μl were added with the following composition:

35% (8.75 μg) p-LC (SEQ ID NO: 1);
35% (8.75 μg) p-HC (SEQ ID NO: 3);
10% (2.5 μg) p-p18 (SEQ ID NO: 5);
10% (2.5 μg) p-p21 (SEQ ID NO: 7);
10% (2.5 μg) p-aFGF (SEQ ID NO: 9).

Plasmid p-LC comprises the genetic information for the production of the light chain of an IgG antibody, plasmid p-HC comprises the genetic information for the production of the heavy chain of an IgG antibody, plasmid p-p18 comprises the genetic information for the production of the protein p18, plasmid p-p21 comprises the genetic information for the production of the protein p21 [5], plasmid p-aFGF comprises the genetic information for the production of the protein aFGF. The particular vector constructs used in the present invention can also be obtained from the inventors. The inventors can be contacted via e-mail at hildinger@gmx.net. Moreover, commercial services do exist that produce any desired nucleotide sequence even comprising several kilobase pairs including complete expression cassettes in plasmid backgrounds (e.g., Invitrogen, Carlsbad, USA; Geneart, Germany). Thus, providing the genetic sequence information of the plasmids should enable one of ordinary skill in the art to order the plasmids of the present invention at one of the commercial services listed.

After shortly mixing the DNA with the cells by gentle shaking, 50 μg of 25-kd linear polyethyleneimine (“PEI”; Polysciences, Eppelheim, Germany; [2]) were added at a concentration of 1 μg/μl. After shortly mixing by gentle shaking, the filter tube containing the cells together with the DNA and PEI was transferred into an orbital shaker (Kühner Shaker Cabinet ISF-4-W, “Kühner shaker”, Kühner AG, Birsfelden, Switzerland), and the cells were incubated for 4 hours at 37° C. in a 5% CO₂ atmosphere under shaking at 180 rpm. After that time, 4.5 ml of Ex-Cell medium comprising 1× antibiotic, antimycotic solution (Sigma Aldrich; A5955; Lot 103K2393) were added. Then, sodium butyrate was added to a final concentration of 3 mmol/l. The cells then were returned into the Kühner shaker and incubated at 37° C. in a 5% CO₂ atmosphere under shaking at 180 rpm. Every seven days post transfectionem, cells were fed with 200 μl of a solution containing 400 g/l glucose and 200 mmol/l L-glutamine.

Each day, 25 μl of supernatant was removed from the cells in order to determine the antibody titer via ELISA. The ELISA was performed as published in prior art [3]. In short, Goat anti-human kappa light chain IgG (Biosource) was used for coating the ELISA-plates, and with AP-conjugated goat anti-human gamma chain IgG (Biosource) the synthesized IgG1 was detected. NPP was used as a substrate for the alkaline phosphatase. Absorption was measured at 405 nm against 490 nm using a microplate reader (SPECTRAmax™340; Molecular Devices, Palo Alto, Calif., USA).

The following results were obtained:

| Day after transfection | Batch yield (mg/l) |
|---|---|
| 0 | — |
| 1 | 28.5 |
| 2 | 69.9 |
| 3 | 144.0 |
| 4 | 219.7 |
| 5 | 291.9 |
| 6 | 365.7 |
| 7 | 420.2 |
| 8 | 485.6 |
| 9 | 539.8 |
| 10 | 582.1 |
| 11 | 617.3 |
| 12 | 645.6 |
| 13 | 670.6 |
| 14 | 684.8 |
| 15 | 697.7 |
| 16 | 707.6 |
| 17 | 712.8 |
| 18 | 714.2 |

As one can see, batch yields exceeding 700 mg/l were obtained at day 16. Moreover, specific productivity >35 pg/cells/day could be achieved from day 3 to day 4.

### PRIOR ART CITATIONS

1. DUROCHER, Y, et al., A reporter gene assay for high-throughput screening of G-protein-coupled receptors stably or transiently expressed in HEK293 EBNA cells grown in suspension culture. Anal Biochem, 2000. 284 (2): p. 316-26.
2. BALDI, L, et al., Transient gene expression in suspension HEK-293 cells: application to large-scale protein production. Biotechnol Prog, 2005. 21 (1): p. 148-53.
3. MEISSNER, P, et al., Transient gene expression: recombinant protein production with suspension-adapted HEK293-EBNA cells. Biotechnol Bioeng, 2001. 75 (2): p. 197-203.
4. STETTLER, M, et al., New disposable tubes for rapid and precise biomass assessment for suspension cultures of mammalian cells. Biotechnol Bioeng, 2006.
5. FUSSENEGGER, M, et al., Controlled proliferation by multigene metabolic engineering enhances the productivity of Chinese hamster ovary cells. Nat Biotechnol, 1998. 16 (5): p. 468-72.

What is claimed is:

1. A method for producing a recombinant protein in a mammalian cell at a specific productivity of at least 35 pg per cell per day, said method comprising introducing nucleic acid molecules encoding the recombinant protein of interest into the mammalian cell by means of transient transfection.

2. A method for producing a recombinant protein in mammalian cells at a batch yield of at least 700 mg/l, said method comprising introducing nucleic acid molecules encoding the recombinant protein of interest into the mammalian cells by means of transient transfection.

3. A method for producing a recombinant protein in a mammalian cell at a specific productivity of at least 20 pg per cell per day, said method comprising introducing nucleic acid molecules encoding the recombinant protein of interest into the mammalian cell by means of transient transfection.

4. A method for producing a recombinant protein in mammalian cells at a batch yield of at least 200 mg/l, said method comprising introducing nucleic acid molecules encoding the recombinant protein of interest into the mammalian cells by means of transient transfection.

5. A method for producing a recombinant protein in mammalian cells at a volumetric yield of at least 2 grams, said method comprising introducing nucleic acid molecules encoding the recombinant protein of interest into the mammalian cells by means of transient transfection.

6. The method of claims 1 to 5 wherein said cells are transfected in serum-free medium.

7. The method of claims 1 to 6 wherein said cells are transfected at a cell density exceeding 5 million cells per ml.

8. The method of claims 1 to 6 wherein said cells are transfected at a cell density of 20 million cells per ml.

9. The method of claims 1 to 6 wherein said cells are transfected at a packed cell volume (PCV) exceeding 2.5.

10. The method of claims 1 to 6 wherein said cells are transfected at a packed cell volume (PCV) of 10.

11. The method of claims 1 to 10 wherein said cells are transfected using 25 kd linear polyethyleneimine as transfection reagent.

12. The method of claim 11 wherein said cells are transfected at a PEI:DNA ratio of 4:1 to 1:1 (weight:weight).

13. The method of claim 11 wherein said cells are transfected at a PEI:DNA ratio of 2:1 (weight:weight).

14. The method of claim 11 wherein said cells are transfected with 5 μg PEI and 2.5 μg DNA per 1 million cells.

15. The method of claim 11 wherein said cells are transfected using 100 μg PEI and 50 μg DNA per 1 ml of medium during the time of transfection.

16. The method of claims 1 to 15 wherein said cells are transfected by directly adding the nucleic acid molecules to the cells and then adding the corresponding amount of transfection reagent.

17. The method of claims 1 to 5 wherein said mammalian cells are kept in serum-free medium after transfection.

18. The method of claims 1 to 5 and 17 wherein said cells are kept at a density of 0.5 million cells per ml to 6 million cells per ml after transfection.

19. The method of claims 1 to 5 and 17 wherein said cells are kept at a density between 1.5 million cells per ml and 4.5 million cells per ml after transfection.

20. The method of claims 1 to 5 wherein said cells are kept in a medium comprising sodium butyrate at a final concentration of 0.5 mmol/l to 8 mmol/l.

21. The method of claims 1 to 5 wherein said cells are kept in a medium comprising sodium butyrate at a final concentration of 3 mmol/l.

22. The method of claims 20 and 21 wherein sodium butyrate is added to the cells 0 hours to 48 hours after transfection.

23. The method of claims 1 to 5 wherein said mammalian cells are incubated at a temperature of 28° C. to 35° C.

24. The method of claims 1 to 5 wherein said mammalian cells are incubated at a temperature of 31° C.

25. The method of claims 1 to 5 wherein said mammalian cell is incubated at a temperature of 37° C.

26. The method of claims 1 to 5 wherein said mammalian cells are kept in a medium comprising 2-aminopurine at a final concentration of 5 mmol/l to 20 mmol/l.

27. The method of claims 1 to 5 and 17 to 26 wherein the nucleic acid molecule encoding the recombinant protein of interest is co-transfected with a nucleic acid molecule encoding one or several of the following proteins: p18, p21 and/or aFGF.

28. The method of claim 27 wherein the nucleic acid molecule encoding the recombinant protein of interest is co-transfected with a nucleic acid molecule encoding p18, where 5% to 30% of the total quantity of DNA transfected is comprised by the nucleic acid molecule encoding p18.

29. The method of claim 27 wherein the nucleic acid molecule encoding the recombinant protein of interest is co-transfected with a nucleic acid molecule encoding p21, where 5% to 30% of the total quantity of DNA transfected is comprised by the nucleic acid molecule encoding p21.

30. The method of claim 27 wherein the nucleic acid molecule encoding the recombinant protein of interest is co-transfected with a nucleic acid molecule encoding p21 and a nucleic acid molecule encoding p18, where 5% to 30% of the total quantity of DNA transfected is comprised by the nucleic acid molecules encoding p18 and p21, respectively.

31. The method of claim 27 wherein the nucleic acid molecule encoding the recombinant protein of interest is co-transfected with a nucleic acid molecule encoding aFGF, where 1% to 15% of the total quantity of DNA transfected is comprised by the nucleic acid molecule encoding aFGF.

32. The method of claim 27 wherein the nucleic acid molecule encoding the recombinant protein of interest is co-transfected with a nucleic acid molecule encoding p21 and a nucleic acid molecule encoding p18 and a nucleic acid molecule encoding aFGF, where 10% of the total quantity of DNA transfected is comprised by the nucleic acid molecules encoding p18, 10% of the total quantity of DNA transfected is comprised by the nucleic acid molecules encoding p21 and 5% of the total quantity of DNA transfected is comprised by the nucleic acid molecules encoding aFGF.

33. The method of claim 27 wherein the nucleic acid molecule encoding the recombinant protein of interest is co-transfected with a nucleic acid molecule encoding p21 and a nucleic acid molecule encoding p18 and a nucleic acid molecule encoding aFGF, where 10% of the total quantity of DNA transfected is comprised by the nucleic acid molecules encoding p18, 10% of the total quantity of DNA transfected is comprised by the nucleic acid molecules encoding p21 and 10% of the total quantity of DNA transfected is comprised by the nucleic acid molecules encoding aFGF.

34. The method of claims 1 to 5 and 27 to 33 wherein said recombinant protein is a secreted protein.

35. The method of claim 34 wherein said secreted recombinant protein is an antibody.

36. The method of claim 34 wherein said secreted recombinant protein is a secreted protein with an FC-fusion tag.

37. The method of claims 1 to 5 wherein said cells are kept in a non-instrumented bioreactor.

38. The method of claim 37 wherein said non-instrumented bioreactor is shaken.

39. The method of claims 37 and 38 wherein said non-instrumented bioreactor is a 20 liter Nalgene bottle or a 50 ml filter tube.

40. The method of claims 1 to 26 and 37 to 39 wherein said mammalian cells are human embryonic kidney cells 293 (HEK 293) or Chinese Hamster Ovary Cells (CHO).

41. The method of claims 1 to 5 wherein suspension adapted mammalian cells undergo the steps of: (1) Adjustment of cell density to a PCV of 10; (2) Mixing of said mammalian cells at a PCV of 10 sequentially with 2.5 μg DNA and 5 μg PEI for each million of cells—with the DNA consisting of plasmids encoding the protein of interest, p18, p21, aFGF and with the plasmids transfected at a (weight-based) ratio of 70:10:10:10; (3) Incubation for 4 hours under shaking; (4) Dilution to a PCV of 2 with medium; (5) Addition of sodium butyrate to a final concentration of 3 mmol/l; (5) Incubation at 37° C. in a 5% CO₂ atmosphere for 18 days under shaking.

42. The method of claims 1 to 5 wherein suspension adapted mammalian cells undergo the steps of: (1) Adjustment of cell density to a PCV of 3 to 10; (2) Mixing of said mammalian cells at a PCV of 3 to 10 with 1 to 5 μg DNA and 1 to 15 μg PEI for each million of cells—with the DNA consisting of plasmids encoding the protein of interest, p18, p21 and aFGF; (3) Incubation for 1.5 to 6 hours under shaking; (4) Dilution to a PCV of 0.5 to 3 with medium; (5) Addition of sodium butyrate to a final concentration of 0.5 to 6 mmol/l within 0 hours to 48 hours after dilution; (5) Incubation at 31° C. to 37° C. in a 0% to 5% CO₂ atmosphere for 5 to 20 days.

43. The method of claims 41 and 42 wherein said adjustment of cell density is made to a cell density of 6 million cells per ml to 20 million cells per ml.

44. The method of claims 41 to 43 where the plasmid encoding aFGF, p18 or p21 is replaced with the plasmid encoding the recombinant protein of interest.

45. The method of claims 41 to 44 where the protein of interest is an antibody and two plasmids are used to express the protein of interest—with one plasmid encoding the heavy chain, the other plasmid encoding the light chain of the antibody of interest and where both plasmids are used in an equimolar ratio.
